(19) United States
(12) Patent Application Publication
(10) Pub. No.: US 2014/0130744 A1
(43) Pub. Date: May 15, 2014

(54) VERMICOMPOSTING METHOD AND APPARATUS

(71) Applicant: GARY TOET, Carmel Valley Village, CA (US)

(72) Inventor: GARY TOET, Carmel Valley Village, CA (US)

(21) Appl. No.: 13/677,301

(22) Filed: Nov. 15, 2012

(51) Int. Cl. A01K 67/033

(52) U.S. Cl. CPC A01K 67/0332
USPC 119/6.7

(57) ABSTRACT

A method and device are provided for transferring heat from decomposing manure to a worm growth zone. A shelter contains manure in a first area and a worm growth bed in a second area. The worm growth bed supports a plurality of worms substantively covered by a layer of manure. Vermicast passes through a supporting grid of the worm growth bed and deposits on a floor or a bottom of the second area. Heat generated by the manure is transferred to the worm growth bed by radiant heat transfer and/or by air convection. The manure is transferred portion by portion over time from the first area to the worm growth bed. The shelter may include a battery, a solar power converter, a microcontroller, dynamic memory, a real-time clock, a wireless transponder, temperature sensors, humidity sensors, water pumps, and/or springs or motors used to open or close apertures of walls of the shelter.

### RELATED APPLICATION

This Application is a Nonprovisional Continuation-in-Part Patent Application of, and claims the benefit of, the filing date of U.S. Provisional Patent Application Ser. No. 61/560,189, filed Nov. 16, 2011 and titled VERMICOMPOSTING METHOD AND APPARATUS and which U.S. Provisional Patent Application Ser. No. 61/560,189 is incorporated herein by reference in its entirety.

### FIELD OF THE INVENTION

The present invention relates to vermiculture. More particularly, the present invention relates to exploiting the use of animal manure to sponsor and harvest worms and vermicompost.

### BACKGROUND OF THE INVENTION

Various methods of vermicomposting apply worms to break down waste materials such as livestock manure. Vermicompost is the product or process of composting utilizing various species of worms, usually red wigglers, white worms, and earthworms to create a heterogeneous mixture of decomposing vegetable or food waste, bedding materials, and vermicast. Vermicast, similarly known as worm castings, worm humus or worm manure, is the end product of the breakdown of organic matter by a species of earthworm.

Most worm species thrive best in the temperature range of 70° F.-80° F., but can survive within an extreme range of 45° F.-90° F. Animal manure sponsors worm yield not only by being a food for worms, but also by generating heat as the manure decomposes.

Yet, the prior art fails to optimally apply the heat generated by decomposing manure to maintain a plurality of worms within an intended temperature range.

### SUMMARY OF THE INVENTION

Toward this and other objects that are made obvious in light of the disclosure, a method and apparatus are provided that transfer heat from decomposing manure to a worm growth zone. In a first aspect of the method of the present invention a shelter is provided that contains manure in a first area and a worm growth bed in a second area. According to another aspect of the method of the present invention, the manure is placed first into the first area and is transferred portion by portion over time to the worm growth bed. The worm growth bed preferably supports a plurality of worms substantively covered by a layer of manure. The worm growth bed comprises an open grid upon which the worms and manure layer are initially positioned and through which the vermicast may pass through the worm growth bed and deposit on a floor or a bottom of the second area.

Heat generated by manure placed in the first area may be transferred to the worm growth bed by radiant heat transfer and/or by air convection. The shelter may be include a fan or other suitable air propulsion device that is applied to encourage transfer of heat from the manure of the first area and to the worm growth bed. Alternatively or additionally, the shelter may be insulated to retain heat and/or include a fan or other suitable air propulsion device positioned to transfer air into and/or out of the shelter in order to regulate the temperature of the worm growth bed in particular and/or the shelter in general.

The shelter may further include a measuring cup and/or a transfer tray that are located within the shelter and are positioned and used to measure and transfer manure for controlled shifting of manure from the first area and to the worm growth bed.

According to another aspect of the method of the present invention, the shelter sponsors the growth and harvesting of both vermicompost and worms, whereby worms may be removed from the shelter for sale, application in agriculture, or placement in an additional vermicompost shelter.

Shelters may be sold, leased or rented. The commercial operation of the shelter may exploit the relative market values of vermicompost, vermicast and worms versus the market value of animal manure. The profits and/or shares of the output of the shelter, e.g., worms and vermicompost, may be divided among two or more contributing parties, such as a party who (a.) builds or provides the shelter or shelter components; (b.) delivers the shelter to a location near manure and/or crops targeted for application of worms or vermicompost; (c.) constructs the shelter; (d.) provides a site for the shelter; (e.) manages the operation of the shelter; (f.) provides electrical energy and/or water to the shelter; and/or (g.) provides or delivers worms and/or manure to the shelter.

The shelter may further comprise a water distribution system to support optimal hydration of the worm growth bed and/or manure contained within the first area. Additionally or additionally, the shelter may include a battery, a solar power converter, a microcontroller, dynamic memory, a real-time clock, a wireless transponder, one or more temperature sensors, one or more humidity sensors, one or more water pumps, and/or springs or motors used to open or close apertures of exterior and/or internal walls of the shelter.

The shelter may additionally be designed to isolate and protect the worms and the manure contained within from damage that can be imposed by environmental factors, disease, infections, birds, pests and vermin.

The shelter may be designed as a modular shelter that includes flat components designed for ease of shipping and construction. The modular shelter may have a removable roof and removable walls that may be detached entirely or in part in order to allow heat to escape from the shelter and to allow access to the manure-holding first area, the worm growth bed and/or to the vermicompost generated in the second area. And external full width of the shelter is optimally less than 8 feet six inches to enable easier transport of the shelter on public highways and roads.

The shelter may be co-located with livestock to reduce the cost and energy expenditure required to deliver the manure to the shelter. For example, the shelter may be located near a horse stables. Furthermore, the shelter may be located on a farm or a vineyard to reduce the cost and energy expenditure required to apply the vermicompost as fertilizer to crops or vines.

The unobvious potential of placing integrated horse manure composters and worm and worm output harvesting structures on site at individual horse properties provides the following benefits:
1. Produces added value to horse manure and other mammalian manure;
2. Can accommodate daily manure pick up of animal output;
3. Can become a manure management handling service business;
4. The service business is created because our business has to tend to the VermiComposter loading, worm feeding, worm harvesting, and output extraction on a regularly scheduled servicing;
5. Invented shelters being located at the horse owner properties reduces business operational cost of property rental, lease or rent, water and electricity;
6. The combination of worms and worm output sales are tied directly to the consistent, professional servicing of each invented shelter sold and placed in the field;
7. A route type service business i.e. a service creates the opportunity to set up distinct geographical independent routes, thus promoting the creation of local green jobs; and
8. Horse owner benefits from eliminating the cost associated with the time, equipment and labor of manure management.

### BRIEF DESCRIPTION OF THE FIGURES

These, and further features of the invention, may be better understood with reference to the accompanying specification and drawings depicting the preferred embodiment, in which:

FIG. 1A is a first perspective view of a shelter;

FIG. 1B is a second perspective view of a shelter;

FIG. 2 is a top cut-away view of the shelter of FIG. 1;

FIG. 3 is a side cut-away view of the shelter of FIG. 1;

FIG. 4 is an external view of the biomass side of the shelter of FIG. 1;

FIG. 5 is an external view of the worm side of the shelter of FIG. 1;

FIG. 6A is a perspective view of the shelter indicating the hinge-enabled mobility of aspects of the shelter of FIG. 1;

FIG. 6B is a perspective view of the shelter indicating the hinge-enabled mobility of aspects of the shelter of FIG. 1;

FIG. 7 is an exploded view of the shelter 2 of FIG. 1 illustrating the modular and configurable design and qualities of the shelter 2;

FIG. 8 is a top view of a worm growth bed of the shelter of FIG. 1;

FIG. 9 is a schematic diagram of an optional control system of the shelter 2 of FIG. 1 and comprising a processor;

FIG. 10 is a diagram of a watering system of the shelter 2 of FIG. 1;

FIG. 11 is a diagram of an optional air ventilation system of the shelter 2 of FIG. 1;

FIG. 12 is a perspective cut-away view showing an optional biomass distribution panel and an optional floor panel assembly of the shelter of FIG. 1;

FIG. 13 is a flowchart of a system software of processor of FIG. 10; and

FIG. 14 is a top view of an alternate bar and winch assembly for use in raking the worm growth bed of FIGS. 1 and 8.

### DESCRIPTION

It is to be understood that this invention is not limited to particular aspects of the present invention described, as such may, of course, vary. It is also to be understood that the terminology used herein is for the purpose of describing particular aspects only, and is not intended to be limiting, since the scope of the present invention will be limited only by the appended claims.

Methods recited herein may be carried out in any order of the recited events which is logically possible, as well as the recited order of events.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the methods and materials are now described.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

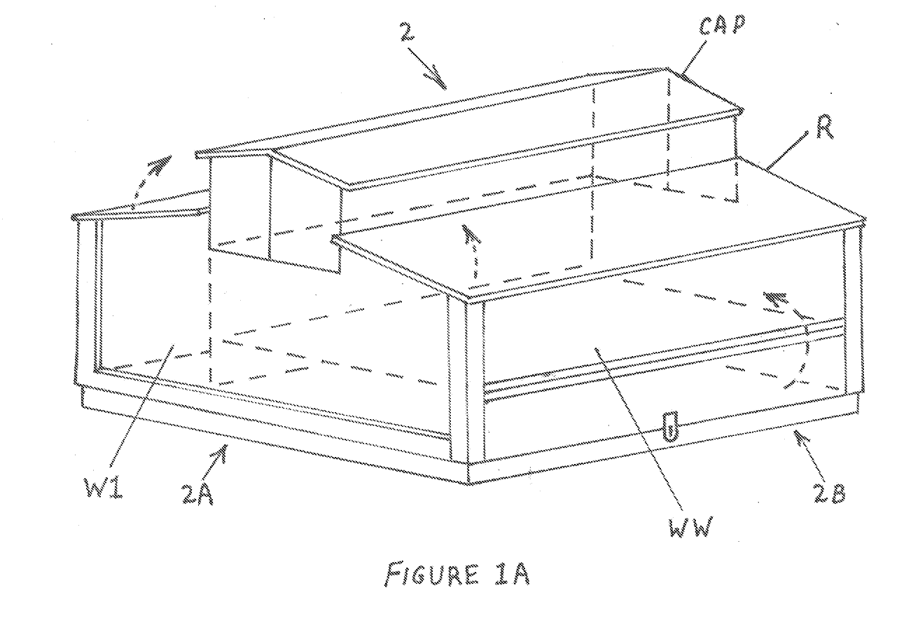

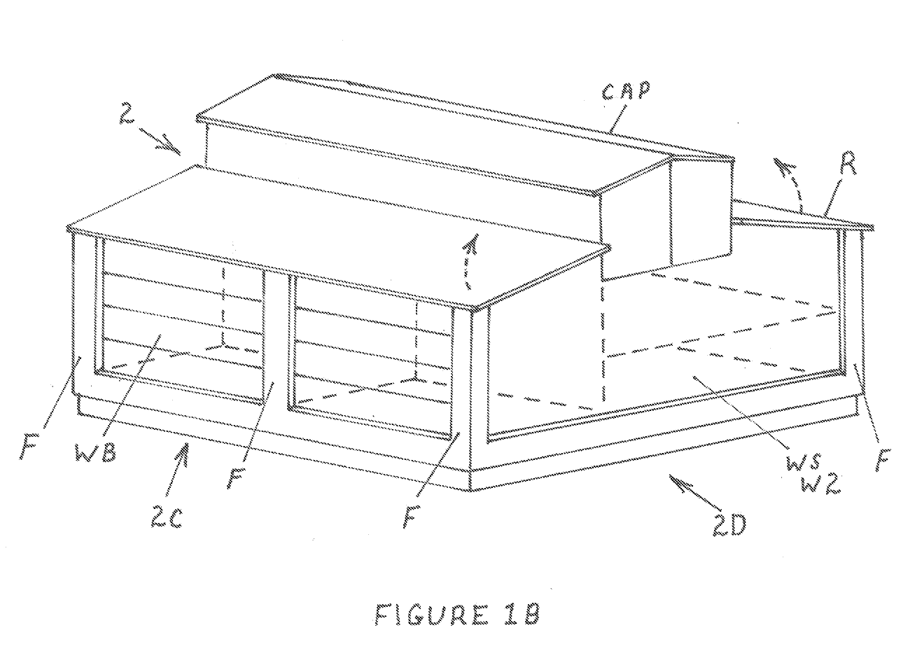

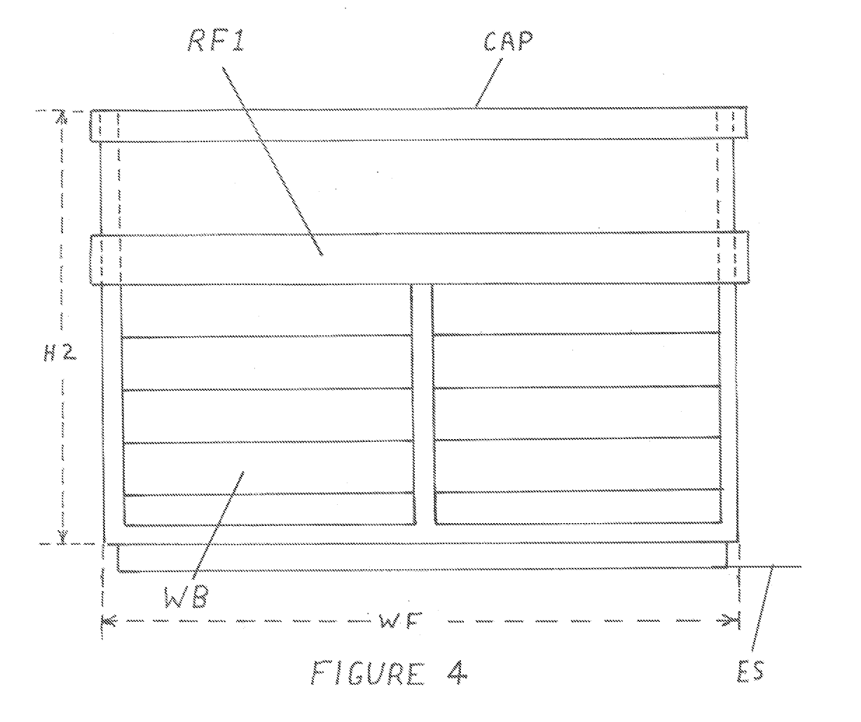

Referring now generally to the Figures and particularly FIG. 1A and FIG. 1B, FIG. 1A is a perspective view of a shelter 2 of a full external width WF as shown on FIG. 4 and a length L, and featuring a first protective side 2A, a worm side 2B and a roof R, whereas FIG. 1B features a biomass side 2C and a second protective side 2D. The shelter 2 further includes framing F, a roof cap CAP, a removable biomass external wall WB, a worm side external wall WW, a first protective wall W1 and a second protective wall W2. The framing F maintains the walls WW, WB, & W1-W9 and roof R in place while allowing hinged or rotatable movement of certain wall elements WW and/or roof sheets RF1 & RF2. The worm wall WW preferably includes two worm section walls W6 & W7 that are separately rotatably and/or hingeably coupled with framing F and/or one or more floor walls W8 & W9.

The total height of the shelter 2, i.e., height to the peak of the roof cap CAP, relative to the earth's surface of the shelter 2 is preferably within the range of 1 foot to 20 feet, and more preferably in the range of from 3 feet to 8 feet.

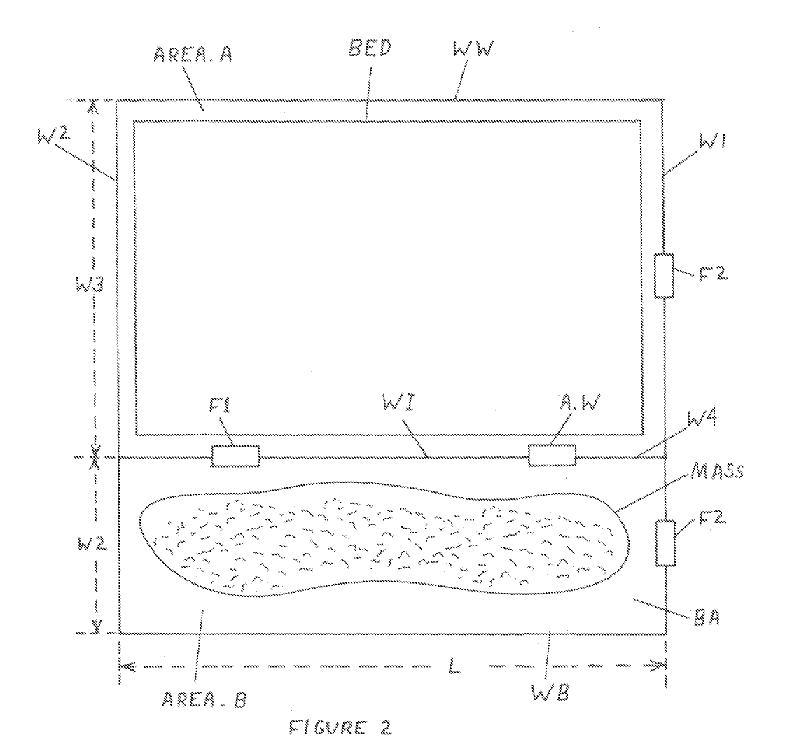

FIG. 2 is a top cut-away view of the shelter 2. A worm growth bed BED is positioned within a worm growth area AREA.A and a formation of biomass MASS, e.g., manure such as horse manure, is positioned within a manure storage area AREA.B located between the biomass external wall WB and an internal wall W4. A worm growth bed BED is positioned between the internal wall W4 and the worm side external wall WW and supports a mass of worms and worm outputs mixed with elements of the biomass MASS, e.g., horse manure, wherein the elements of the biomass MASS located in the worm growth area AREA.A has preferably transferred from the biomass area AREA.B. The roof R, the first protective wall W1 and the second protective wall W2 partially shelter 2 the biomass MASS and the worm growth bed BED from exposure to environmental factors causing environmental degradation, e.g., exposure to inclement weather, precipitation, temperature extremes and precipitation.

The manure storage area AREA.B has (1.) a length L preferably in the range of from 1 foot to 100 feet, and more preferably in the range of from 2 feet to 6 feet; (2.) a manure area width W2 in the range of from 1 foot to 100 feet, and more preferably in the range of from 2 feet to 6 feet; and (3.) a maximal height preferably in the range for 2 feet to 20 feet.

The worm growth area AREA.A has (1.) a length preferably in the range of from 1 foot to 100 feet, and more preferably in the range of from 2 feet to 6 feet: (2.) a width W3 in the range of from 1 foot to 100 feet, and more preferably in the range of from 2 feet to 6 feet; and (3.) a maximal height preferably in the range for 2 feet to 20 feet.

The biomass wall WB may comprise wood and has (1.) a length preferably equal to the length L of the manure storage area AREA.B and within the range of from 1 foot to 100 feet, and more preferably in the range of from 2 feet to 20 feet: and (2.) a wall width preferably in the range of from 1/16 inch to 2 inches, and more preferably in the range of from 0.5 inches one inch. The worm side external wall WW may comprise wood and has (1.) a length preferably equal to the length L of the manure storage area AREA.B and in the range of from 1 foot to 100 feet, and more preferably in the range of from 2 feet to 6 feet: and (2.) a width preferably in the range of from 1/16 inch to 2 inches, and more preferably in the range of from 0.5 inches one inch.

The internal wall W4 allows heat to pass through itself and from the biomass area to the worm growth bed BED and preferably does not extend fully to the roof R, whereby air may carry heat from the biomass MASS to worm growth bed BED. An optional aperture A.W of the internal wall W4 permits heated air to transfer between the biomass area AREA.B and the worm growth bed BED. An optional internal motorized fan F1 may be applied to drive air heated by the biomass through an internal fan aperture of the internal motorized fan F1 and thereby through the internal wall WI aperture.

An optional window WN1 of the shelter 2 allows air to transfer in and out of the shelter 2. An optional second motorized fan F2 is selectively positioned and applicable to drive air into or alternately from the shelter 2 through a second internal fan aperture of the second motorized fan F2.

The worm growth area AREA.A is formed by the internal wall W4, the worm wall WW, the first protective wall W 1 and the second protective wall W2.

The biomass area AREA.B is formed by the internal wall W4, removable biomass external wall WB, the roof R, the first protective wall W1 and the second protective wall W2. The biomass MASS is thus sheltered within the biomass area AREA.B and therein protected from environmental factors, disease, infections, birds, pests and vermin that might damage the biomass area AREA.B is or worms and worm outputs.

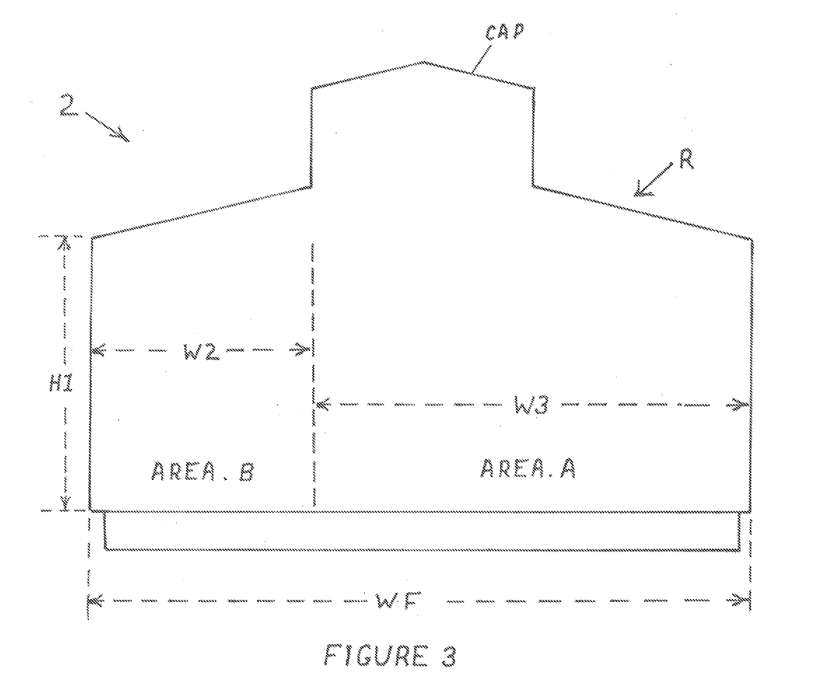

FIG. 3 is a side cut-away view of the shelter 2 showing the worm wall WW and the and the manure storage wall WB having (1.) a wall heights H1 preferably in the range of from 1 foot to 100 feet, and more preferably in the range of from 2 feet to 6 feet; and (2.) widths preferably in the range of from 1/16 inch to 2 inches, and more preferably in the range of from 0.5 inches one inch.

FIG. 4 is an external view of the biomass side showing the biomass wall WB, a first roof sheet RF1 and the roof cap CAP. The roof cap CAP is maximally positioned above the earth's surface ES upon which the shelter sits at a maximal external roof height H2.

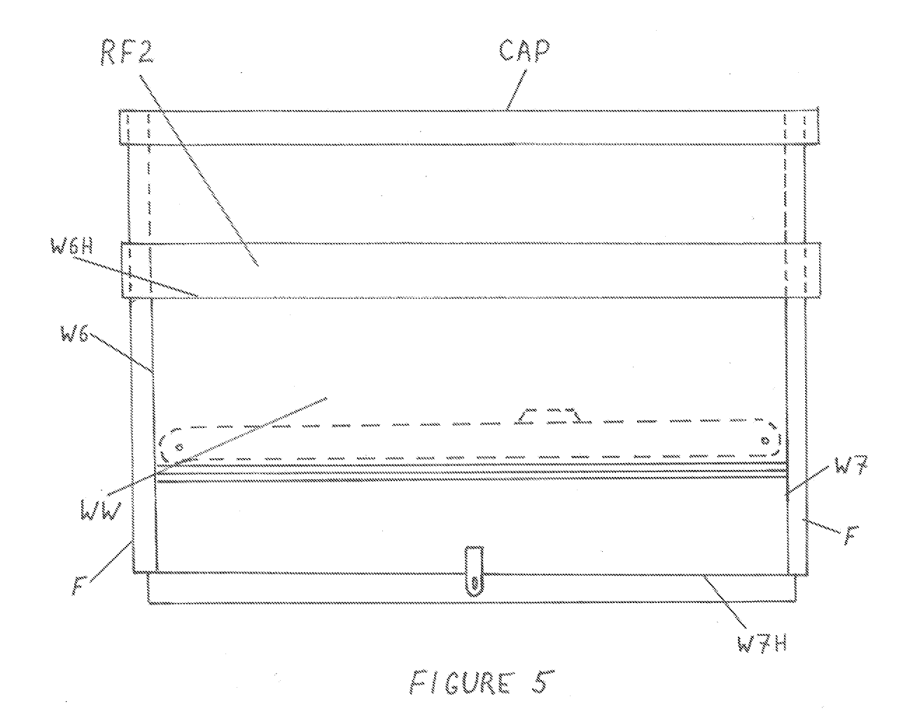

FIG. 5 is an external view of the worm side showing the worm side external wall WW, a second roof sheet RF2 and the roof cap CAP. An upper hinge element W6H is coupled to both (a.) the upper section worm wall W6; and (b.) the framing F. The upper hinge element W6H rotatably couples the upper section worm wall W6 with the framing F.

A lower hinge element W7H is coupled to both (a.) the lower section worm wall W7; and (b.) the framing F and/or one or both floor walls F8 & F9. The lower hinge element W7H rotatably couples the lower section worm wall W7 with the framing F.

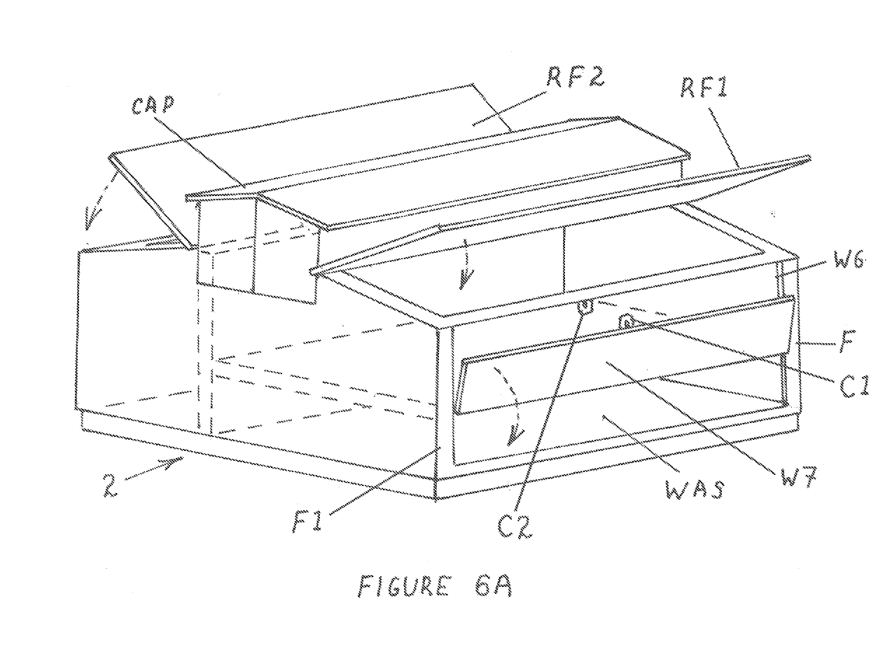

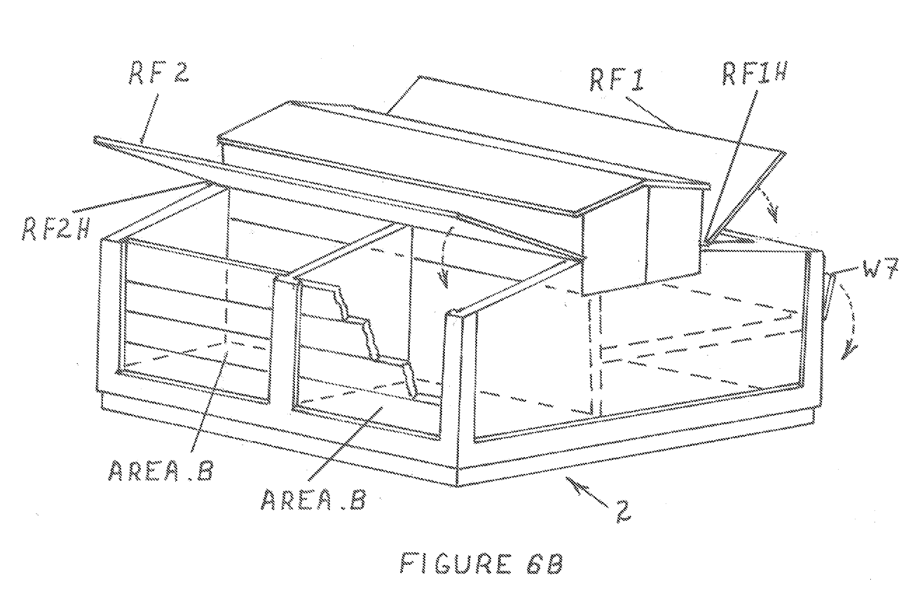

FIG. 6A is a perspective view of the shelter 2 indicating the hinge-enabled mobility of the first roof side RF1, the second roof side RF2, and a worm output access sheet WAS that comprises the lower section worm wall W7, the lower hinge element W7H and a first clasp element C1. The first clasp element C1 is selected to be adapted for repeatedly coupling and decoupling with a second clasp element C2, wherein the first clasp element C1 and the second clasp element C2 enable a detachable coupling of the upper section worm wall W6 and the lower section worm wall W7.

The worm output access sheet WAS may comprise wood having (1.) a length preferably in the range of from 1 foot to 100 feet, and more preferably in the range of from 2 feet to 6 feet: and (2.) a width preferably in the range of from 1/16 inch to 2 inches, and more preferably in the range of from 0.5 inches one inch.

FIG. 6B is a perspective view of the shelter 2 indicating the hinge-enabled mobility of the first roof side RF1, the second roof side RF2, and two manure storage areas. A first upper hinge element RF1H rotatably couples the first roof sheet RF1 with the framing F, and a second upper hinge element RF2H rotatably couples the second roof sheet RF2 with the framing F.

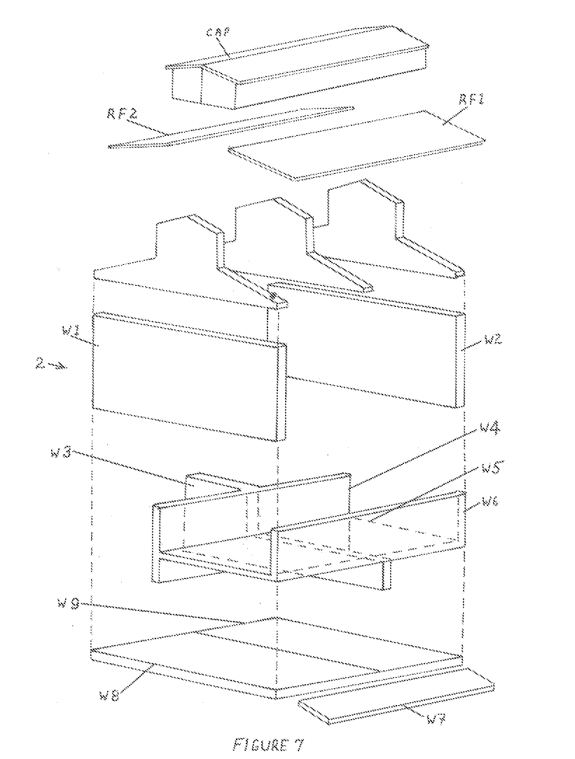

FIG. 7 is an exploded view of the shelter 2 illustrating the modular and configurable design and qualities of the shelter 2, wherein walls W1 through W9 & WAS, roof sheets RF1 & RF2 roof cap CAP, hinge elements W6H, W7H, RF1H & RF2H are adapted for shipment and on-site assembly.

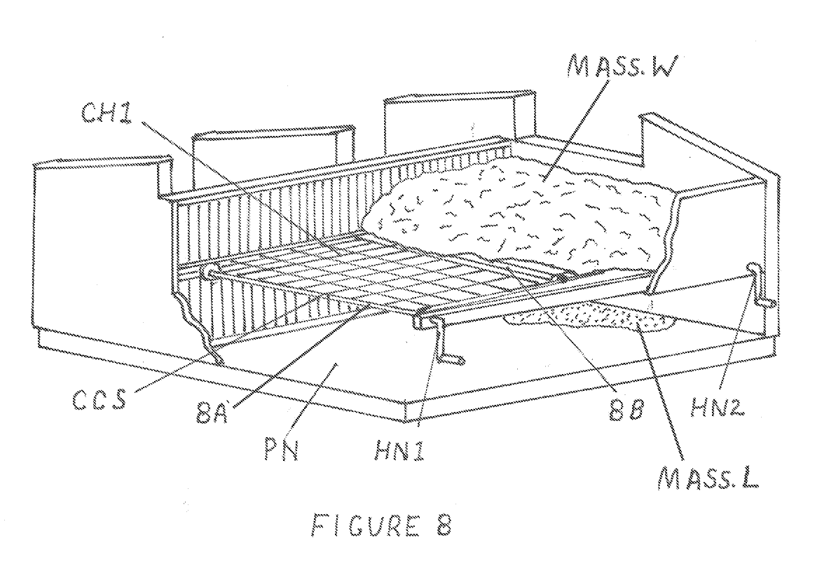

FIG. 8 is a top view of the worm growth bed BED of the shelter 2 and presenting a mass of worm mass MASS.W, a supportive mesh grid 8A positioned horizontally to support the worm mass MASS.W, a bar 8B, and a chain and crank system CCS adapted to drive the bar 8B comb an underside of the worm mass MASS.W. The chain and crank system CCS includes two chains CH1 & CH2 that each (a.) run parallel to the length L of the shelter 2; and (b.) are coupled to opposite ends of the bar 8B.

A first geared hand crank geared HN1 of the chain and crank system CCS is coupled to both chains and adapted to drive the bar 8B from the first protective wall W1 to the second protective wall W2; and a second geared hand crank HN2 of the chain and crank system CCS is coupled to both chains CH 1 & CH2 and adapted to drive the bar 8B from the second protective wall W2 to the first protective wall W1. The movement of the bar 8B to and from the protective walls W1 & W2 and above the supportive mesh grid 8A encourages the worm mass MASS.W to fall through the supportive mesh grid 8A and to form a harvested lower worm mass MASS.L that rests upon the floor walls W8 & W9 and below the supportive mesh grid 8A. The chain and crank system CCS is preferably adapted to permit manual rotation of the geared hand cranks HN1 & HN2 to effectively move the bar 8B.

It is understood that the worm mass MASS.W includes worms, worm outputs, and elements of the biomass MASS. It is further understood that the lower harvested lower worm mass MASS.L includes worms, worm outputs, and elements of the biomass MASS, but usually a lower density of worms than is found in the worm mass MASS.W, as the movement of the bar typically causes relatively more of the worm outputs and the biomass MASS to fall through the supportive mesh grid 8A and to form the harvested lower worm MASS.L than the worms.

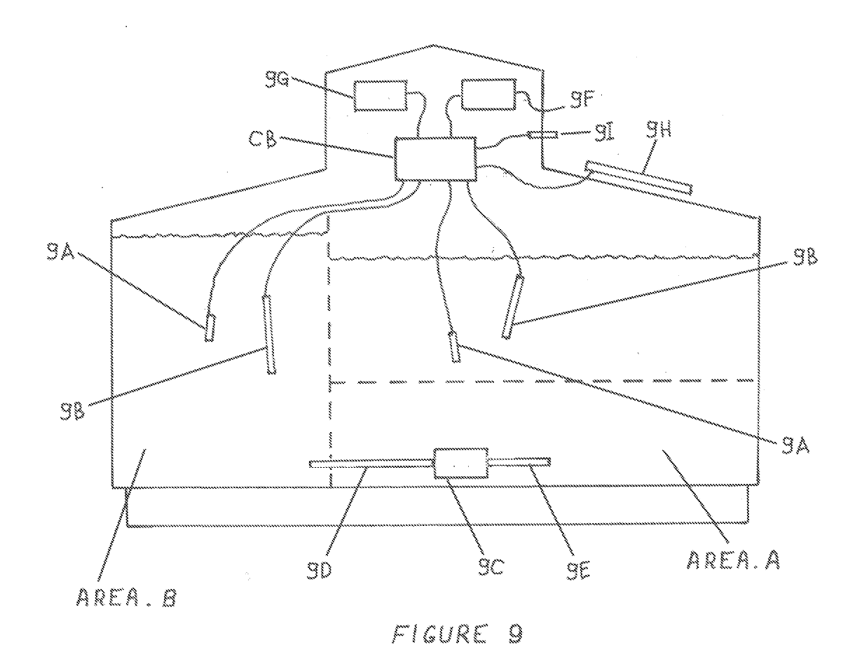

FIG. 9 is a schematic diagram of an optional control system CS of the shelter 2. The control system CS includes a processor CB, wherein the processor CB may include a real time clock and cache memory and may be or comprise. (a.) a network-communications enabled THINKSTATION WORKSTATION™ notebook computer marketed by Lenovo, Inc. of Morrisville, N.C.; (b.) a NIVEUS 5200 computer workstation marketed by Penguin Computing of Fremont, Calif. and running a LINUX™ operating system or a UNIX™ operating system; (c.) a network-communications enabled personal computer configured for running WINDOWS XP™, VISTA™ or WINDOWS 7™ operating system marketed by Microsoft Corporation of Redmond, Wash.; (d.) a MACBOOK PRO™ personal computer as marketed by Apple, Inc. of Cupertino, Calif.; (e.) an IPAD™ tablet computer as marketed by Apple, Inc. of Cupertino, Calif.; (f.) an IPHONE™ cellular telephone as marketed by Apple, Inc. of Cupertino, Calif.; (g.) an HTC TITAN II™ cellular telephone as marketed by AT&T, Inc. of Dallas, Tex. and running a WINDOWS 7™ operating system as marketed by Microsoft Corporation of Redmond, Wash.; (h.) a GALAXY NEXUS™ smart phone as marketed by Samsung Group of Seoul, Republic of Korea or and running an ANDROID™; (i.) a TOUGHPAD™ tablet computer as marketed by Panasonic Corporation of Kadoma, Osaka, Japan and running an ANDROID™ operating system as marketed by Google, Inc. of Mountain View, Calif.; or (j.) other suitable computational system or electronic communications device known in the art.

The control system CS may include the processor CB, electronic memory, an optional battery and/or an external power line, one or more temperature sensors 9A, one or more motorized fans F1 & F2, one or more humidity sensors 9B, a water pump 9C, water delivery lines 9D & 9E, a water source (not shown), a window servomotor 9F, a real time clock, a wireless communications transceiver, and a display device are coupled to the processor via a system communications and power network COMMS. A battery 9G may be coupled to one or more solar energy panels 9H and may provide electrical power to the control system CS. Alternatively or optionally, one or more solar energy panels 9H may provide electrical power directly to the control system CS. An external temperature sensor 9I of the control system CS provides digitized measurements of the temperature external to the shelter 2.

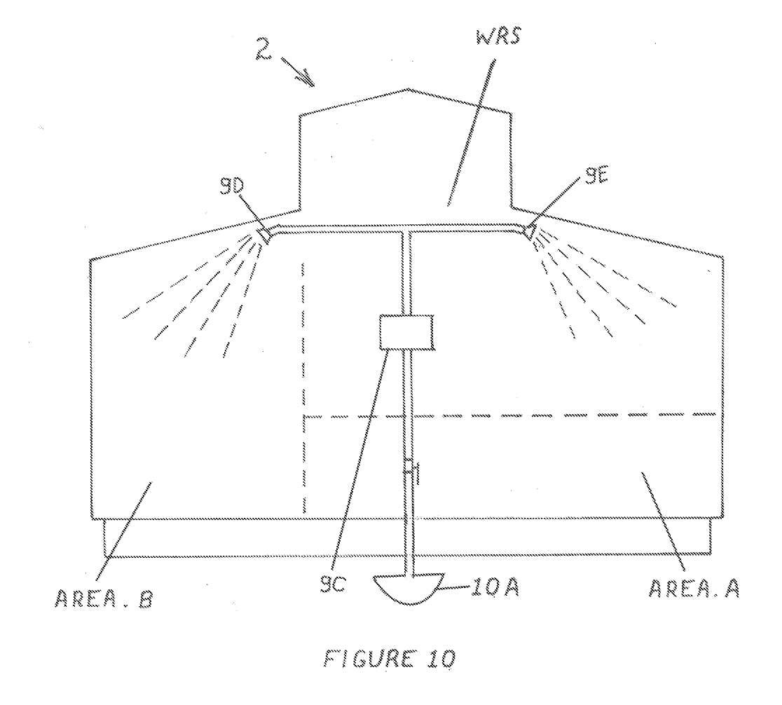

FIG. 10 is a diagram of a watering system of the shelter 2. Tubing leads 9D & 9E lead from a water source 10A and to the biomass MASS and the worm growth bed BED and enabled by the water pump 9C. The water may be driven by into the shelter 2 by pressure provided by the water source 10A and/or optionally by the optional water pump 9C.

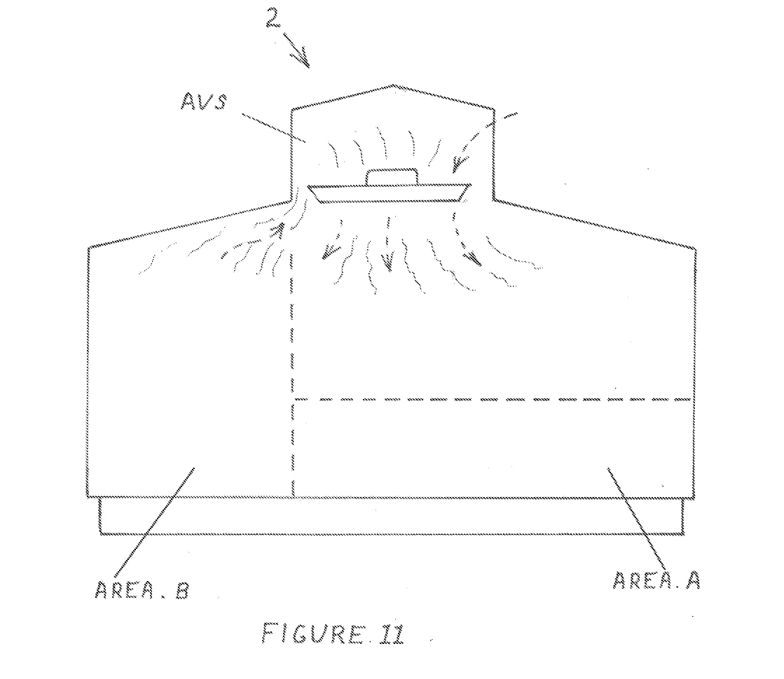

FIG. 11 is a diagram of an optional air ventilation system of the shelter 2 of FIG. 1. One or more motorized fans are coupled to the energy source and optionally to a manual power switch, a timer and/or a controller. The power source may be a power line and and/or a battery. The timer may be configured to cause the fans to periodically energize and transfer air. Alternatively or additionally, the controller may be programmed to cause the fans to periodically energize and transfer air on a periodic basis or an event driven basis.

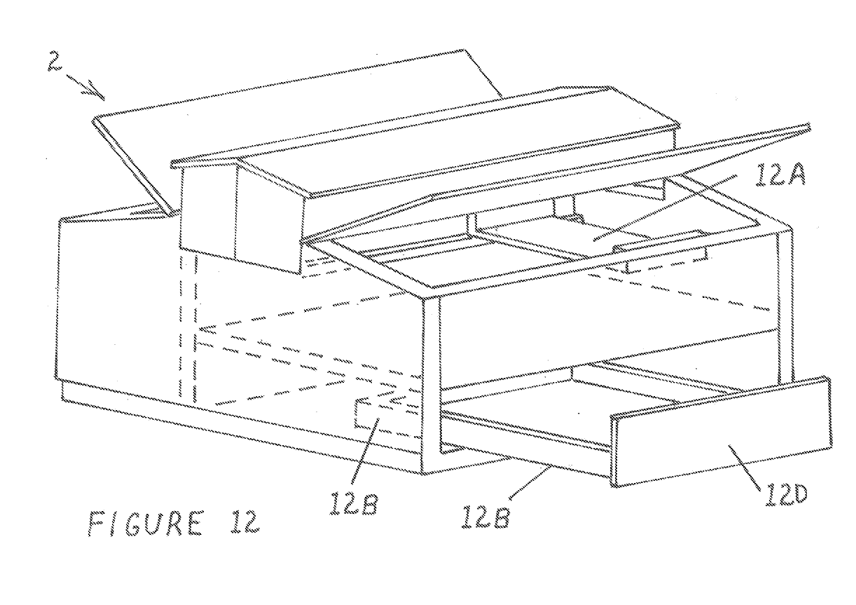

FIG. 12 is a perspective cut-away view showing an optional biomass distribution panel 12A and a floor panel assembly 12B. The floor panel assembly 12B presents a roller module 12C that enables a floor panel 12D to be positioned beneath both the supportive mesh grid 8A and the worm mass MASS.W and to capture the harvested lower worm mass MASS.L. The roller module 12C further enables the floor panel 12D to be pulled away from the inner wall W4 to allow easier to access the harvested lower worm mass MASS.L and to remove the harvested lower worm mass MASS.L from the shelter 2. The roller module 12C additionally enables the floor panel 12D to be pushed toward the inner wall W4 to allow easy positioning centrally below the worm mass MASS.W and to allow accumulation of the harvested lower biomass MASS.L.

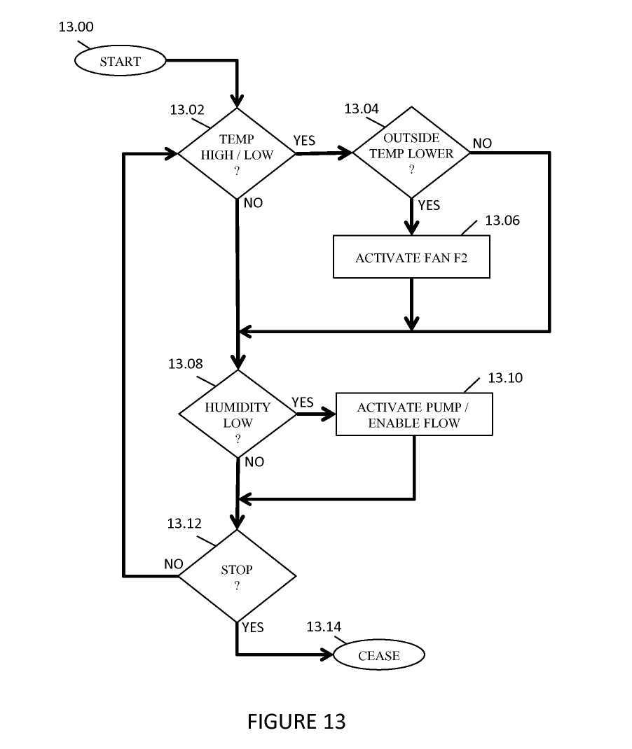

Referring now generally to the Figures and particularly to FIG. 13, FIG. 13 is a flowchart of a system software of the control system CS. The control system CS reads the internal temperature of the shelter 2 from the internal temperature sensors 9A and determines if the temperature is too high or too low and if the temperature is too high, i.e., above a pre-established set point T1, then determines in step 1304 if the temperature measurement of the external temperature sensor 9I indicates that the ambient temperature outside of the shelter is higher than the temperature measurement of the internal temperature sensors 9A indicates. When the temperature measurement of the external temperature sensor 9I of step 1304b indicates that the ambient temperature outside of the shelter is lower than the temperature measurement of the internal temperature sensors 9A indicates, then the processor CB activates the external fan F2 to drive air from outside the shelter 2 into the shelter 2 in step 1306.

The processor CB next accepts a humidity measurement from a humidity sensor 9B in step 1308 and when determining that the humidity reading is below a prespecified value, activates the pump 9C in step 1310 to drive water through the tubing 9D & 9E and/or merely allows water to flow freely from the water source 10A. The processor CB then determines in step 1312 whether to perform another cycle of step 1302 through 1312 or to halt or cease operations and perform alternate operations in step 1314.

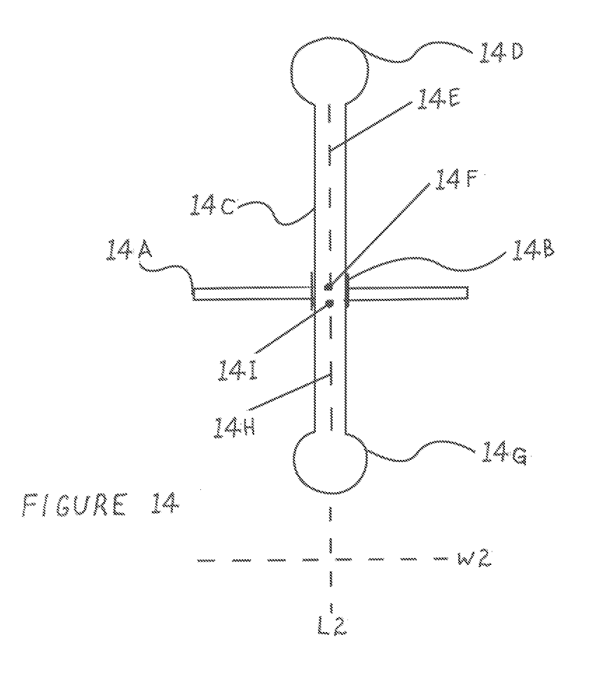

Referring now generally to the Figures and particularly to FIG. 14, FIG. 14 is a top view of an alternate bar and winch assembly for use in raking the worm growth bed. An alternate bar 14A includes a U fixture 14B which is adapted to allow motion along a length axis L2 of a guide bar that is orthogonal to a width axis W2 of the alternate bar 14A and parallel with the length L of the shelter 2. A first manual winch 14D includes a first cable 14E that is coupled to the alternate bar 14A at a first bar attachment point 14F. A second manual winch 14G includes a second cable 14H that is coupled to the alternate bar 14A at a second bar attachment point 14I. The first winch 14D is positioned within the worm growth bed BED and adapted to drive the alternate bar 14A from the first protective wall W1 to the second protective wall W2; and second winch 14G is positioned within the worm growth bed BED and adapted to drive the alternate bar 14A from the second protective wall W2 to the first protective wall W1.

The foregoing disclosures and statements are illustrative only of the Present Invention, and are not intended to limit or define the scope of the Present Invention. The above description is intended to be illustrative, and not restrictive. Although the examples given include many specificities, they are intended as illustrative of only certain possible configurations or aspects of the Present Invention. The examples given should only be interpreted as illustrations of some of the preferred configurations or aspects of the Present Invention, and the full scope of the Present Invention should be determined by the appended claims and their legal equivalents. Those skilled in the art will appreciate that various adaptations and modifications of the just-described preferred embodiments can be configured without departing from the scope and spirit of the Present Invention. Therefore, it is to be understood that the Present Invention may be practiced other than as specifically described herein. The scope of the present invention as disclosed and claimed should, therefore, be determined with reference to the knowledge of one skilled in the art and in light of the disclosures presented above.

What is claimed is:

1. A shelter comprising:
a. a structure comprising a first area and a second area;
b. a heat generating biomass positioned within the first area; and
c. a plurality of live worms positioned within the second area, whereby the structure enables a transfer of heat from the biomass to the plurality of worms.

2. The shelter of claim 1, wherein the biomass nutritionally supports the plurality of live worms.

3. The shelter of claim 1, wherein the biomass comprises manure.

4. The shelter of claim 1, wherein the biomass comprises horse manure.

5. The shelter of claim 1, where the frame comprises a wall disposed between the biomass and plurality of live worms, and the wall is comprised within means to transfer heat from the biomass to the plurality of live worms through heat convection.

6. The shelter of claim 1, wherein a biomass volume is sourced from the biomass positioned within the second area.

7. The shelter of claim 6, wherein the biomass volume is manure.

8. The shelter of claim 1, further comprising a motorized fan positioned to transfer heat from the biomass to the plurality of live worms.

9. The shelter of claim 8, wherein the motorized fan is disposed above the biomass and the plurality of live worms.

10. The shelter of claim 8, wherein the fan is electrically powered.

11. The shelter of claim 1, further comprising a ventilation window.

12. The shelter of claim 11, wherein a flow area of air circulation of the ventilation window is adjustable by movement of the ventilation window.

13. The shelter of claim 1, further comprising a water delivery tubing, the water delivery tubing adapted to provide water to the biomass.

14. The shelter of claim 1, further comprising a water delivery tubing, the water delivery tubing adapted to provide water to the plurality of live worms.

15. A system, the system comprising:
a heat generating biomass;
a plurality of live worms;
a control system comprising:
a temperature sensor;
a processor coupled with the temperature sensor;
a display device coupled with the processor, whereby the display device renders a representation of the output of the temperature sensor.

16. The system of claim 15, wherein the temperature sensor is positioned to measure air temperature within the.

17. The system of claim 15, wherein the temperature sensor is positioned to measure a temperature proximate to the plurality of live worms.

18. The system of claim 15, further comprising a motorized fan coupled with the processor, wherein the processor selectively engages the fan to affect a temperature measurement of the temperature sensor.

19. The system of claim 15, further comprising:
a water pump, the water pump coupled with the processor;
a water tubing, the water tubing coupled with water pump and positioned to deliver water to the biomass; and
a water source coupled to the water tubing, whereby the processor selectively directs the water pump to deliver water to the biomass.

20. A method comprising:
a. Forming a heat generating biomass;
b. Grouping a plurality of worms;
c. Transferring a portion of the biomass to feed the plurality of worms; and
d. Transferring heat from the biomass to the plurality of worms.
